(19) United States
(12) Patent Application Publication
(10) Pub. No.: US 2023/0181364 A1
(43) Pub. Date: Jun. 15, 2023

(54) OPTICAL SYSTEM FOR OBTAINING SURGICAL INFORMATION

(71) Applicant: Alcon Inc., Fribourg (CH)

(72) Inventors: John Park, Irvine, CA (US); Qing Xiang, Irvine, CA (US); Ravi Durvasula, Irvine, CA (US); Ronald T. Smith, Irvine, CA (US)

(21) Appl. No.: 18/062,639

(22) Filed: Dec. 7, 2022

Related U.S. Application Data
(60) Provisional application No. 63/265,165, filed on Dec. 9, 2021.

(51) Int. Cl. A61F 9/008; A61F 9/007

(52) U.S. Cl. CPC A61F 9/008; A61F 9/00763; A61F 2009/00897; A61F 2009/00844

(57) ABSTRACT

In certain embodiments, an optical system for obtaining surgical information includes a probe housing a first optical fiber, a light source, a photoanalyzer, and an optical circulator optically coupled to each of the first optical fiber, the light source, and the photoanalyzer. The optical circulator has a first port configured to receive source light generated from the light source, a second port configured to transmit the source light from the first port to the first optical fiber, and a third port configured to transmit return light in the first optical fiber from the second port to the photoanalyzer. The first optical fiber is configured to emit at least a portion of the source light in the first optical fiber from the probe to contact a body structure, and collect light returning from the body structure as a result of the portion of the source light contacting the body structure.

### BACKGROUND

Laser and/or illumination probes may be used during a number of different medical procedures and surgeries. For example, a laser probe may be used during retinal laser surgery in order to seal retinal tears. An illumination probe may be used to provide illumination to a desired location during a procedure, and may be used in combination with a laser probe. In fact, laser and illumination functions may be carried out by separate probes, or they may be combined into a single illuminated laser probe. In either case, laser and/or illumination light is typically transmitted from a laser and/or illumination light source through an optical fiber.

Surgical procedures are often performed primarily based on pre-operative planning as well as the surgeon's past experience. Visualization and data acquisition during surgery remain somewhat limited. For example, existing systems lack the capability to provide the surgeon with information measured in situ related to spectral parameters of tissues/structures and/or treatment results that could enable real-time adjustment of treatment parameters, e.g., laser power, pulse duration or frequency, etc.

Accordingly, what is needed in the art are improved devices for obtaining surgical information during procedures including an improved laser and/or illumination probe.

### SUMMARY

The present disclosure generally relates to devices for obtaining medical information, and more particularly, to an optical system for surgical procedures and methods of use thereof.

In certain embodiments, an optical system for obtaining surgical information is provided. The optical system includes a probe housing a first optical fiber, a light source, a photoanalyzer, and an optical circulator optically coupled to each of the first optical fiber, the light source, and the photoanalyzer. The optical circulator has a first port configured to receive source light generated from the light source, a second port configured to transmit the source light from the first port to the first optical fiber, and a third port. The first optical fiber is configured to emit at least a portion of the source light in the first optical fiber from the probe to contact a body structure (e.g., an eye, ear, nose, throat or other body structure), and collect light returning from the body structure as a result of the portion of the source light contacting the body structure. The third port is configured to transmit the return light in the first optical fiber from the second port to the photoanalyzer. The photoanalyzer is configured to determine one or more spectral parameters of the body structure based on the return light.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a system for providing an illumination light and/or a laser light to a surgical target.

FIG. 2A illustrates an example optical system for obtaining ophthalmic information, according to certain embodiments.

FIG. 2B illustrates another example optical system for obtaining ophthalmic information, according to certain embodiments.

FIG. 2C illustrates an example optical circulator of the optical system of FIG. 2A or FIG. 2B, according to certain embodiments.

FIG. 2D illustrates an example photoanalyzer of the optical system of FIG. 2A or FIG. 2B, according to certain embodiments.

FIG. 3 illustrates an example method for using ranging data obtained by the optical system of FIG. 2A to improve safety during vitrectomy procedures, according to certain embodiments.

FIG. 4 illustrates another example method for using ranging data obtained by the optical system of FIG. 2A to provide constant surface illuminance during a surgical procedure, according to certain embodiments.

FIG. 5 illustrates yet another example optical system for obtaining ophthalmic information, according to certain embodiments.

FIG. 6 illustrates an example probe that may be used herein, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally relates to devices for obtaining medical information, and more particularly, to an optical system for surgical procedures and methods of use thereof.

Certain aspects of the present disclosure provide optical systems for use in obtaining surgical information (e.g., ophthalmic parameters of eye tissues/structures related to treatments and/or disease states, fluid composition inside the eye, hyperspectral/multispectral graphs that indicate absorption and scattering of different wavelengths, etc. with respect to a patient's eye). Other surgical information is also contemplated (e.g., parameters for structures in the ear, nose, throat, etc.). As used herein, the terms “information” and “data” may be used interchangeably to refer to qualitative observations and/or quantitative data. Optical systems described herein may integrate with various existing laser and/or illumination probes, thus benefitting from and expanding upon existing surgical technology platforms and equipment. Optical systems described herein may integrate with each of the various probes without modification to the probe itself, thus providing a cost-effective approach to leveraging existing surgical devices. In addition, optical systems described herein may integrate with surgical consoles, slit lamps, and other microscopes, as well as with other imaging devices.

While optical systems described herein may be used to obtain ophthalmic information from multiple different regions of the eye, it is to be understood that the principles of the disclosure can be used on other structures such as the ear, nose, throat, etc.). In certain embodiments, a laser and/or illumination probe may be inserted into the posterior chamber of the eye to obtain information related to the back of the eye, which may be used to improve the diagnosis and/or treatment of retinal diseases and other conditions affecting the back of the eye.

In certain embodiments, the probe may be used outside the eye to obtain information related to the front of the eye, which may be used to improve the diagnosis and/or treatment of dry eye, among other conditions affecting the front of the eye. In certain embodiments, information obtained from the front of the eye is valuable for determining cataract grading to help assess cataract progression. Advantageously, use of the probe outside the eye enables enhanced data acquisition even in clinical settings.

In general, information obtained using optical systems and/or methods described herein may be valuable in multiple types of procedures such as retinal surgery, cataract surgery, diagnostic procedures (e.g., diagnosis of dry eye and glaucoma), and other ophthalmic procedures as well as in the detection of disease conditions (e.g., retinal blastoma). For example, in certain embodiments, the information may indicate a distance between a laser and/or illumination probe and the eye wall, which may be used to help prevent laser-induced tissue damage or tissue damage caused by physical contact, thereby improving laser safety. In certain embodiments, the information may be used for laser titration. During laser titration according to methods set for herein, laser light absorption in the retina is estimated based on laser light reflection, and optical power of the therapeutic laser is adjusted based on distance between the laser probe and the eye wall in order to provide more consistent laser treatment.

In certain embodiments, information obtained using optical systems and/or methods described herein may relate to one or more parameters of an eye tissue/structure that is undergoing laser treatment, which may be used to adjust a power, pulse duration, pulse frequency, or treatment time of the laser treatment, thereby personalizing the laser treatment and/or improving treatment results.

In certain embodiments, information obtained using optical systems and/or methods described herein may indicate a composition of fluid inside the eye, which may provide additional information to the surgeon regarding the operating environment and/or may be used to adjust one or more fluid parameters, thereby improving the safety and/or effectiveness of a surgical procedure.

In most retinal cases, the surgeon will perform a core vitrectomy to remove the vitreous from the back of the eye. During core vitrectomy, balanced salt solution (BSS) may be used as a liquid filler inside the eye. Since the vitreous is transparent, there is often some uncertainty as to whether all the vitreous is removed. To address this, information obtained using optical systems and/or methods described herein may be used to determine whether a laser and/or illumination probe is disposed in BSS or vitreous, thus providing an indication of whether all the vitreous is removed.

In certain embodiments, information obtained using optical systems and/or methods described herein may relate to one or more parameters of an eye tissue/structure that are used in disease diagnosis and/or stage evaluation, thereby providing additional data points to improve diagnostic accuracy.

In certain embodiments, information obtained using optical systems and/or methods described herein may include hyperspectral/multispectral graphs that indicate which wavelengths of light are being absorbed and which wavelengths are scattered to detect key spectral signatures associated with certain disease conditions such as retinal blastoma.

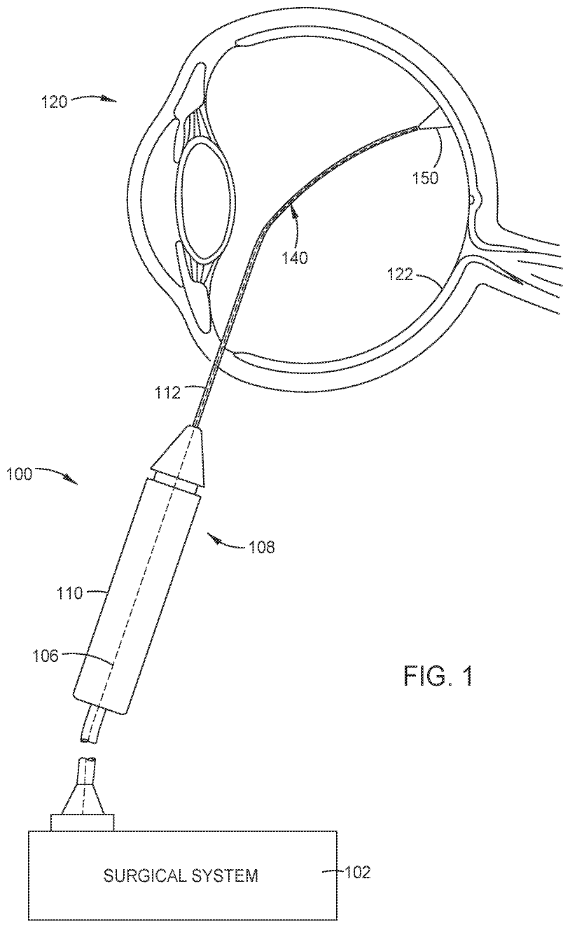

To obtain the ophthalmic information utilized in the example use cases described above, the disclosed optical systems are configured to analyze light returning from an eye structure (referred to as “return light” or “backward light”) as a result of a laser and/or illumination light being projected onto a desired location/surface of the eye during a surgical procedure. As used herein, the term “return light” may include reflection, scattering, fluorescence, auto fluorescence, Raman spectra, or combinations thereof. For example, as shown in FIG. 1, the return light includes a portion of light that is reflected off a retinal surface of the eye and collected in an optical fiber. Conventional systems lack any capability to analyze the return light. Thus, the return light is simply transmitted back towards the light source and eventually lost. Optical systems and/or methods that are configured to utilize the return light are described in more detail below.

FIG. 1 illustrates a system 100 for providing an illumination light and/or a laser light to a surgical target. As shown, system 100 includes a surgical system 102 and a probe 108. Surgical system 102 may include one or more light sources (e.g., laser and/or illumination light sources) for generating laser light beams and/or illumination light beams that may be used during an ophthalmic procedure. For example, the light sources may alternatively, sequentially, or simultaneously generate a laser light beam and an illumination light beam. A user, such as a surgeon or surgical staff member, may control surgical system 102 (e.g., via a foot switch, voice commands, etc.) to emit the laser light beam and/or the illumination light beam during an ophthalmic procedure, such as vitreoretinal surgery. In certain embodiments, surgical system 102 includes a port, and the laser and/or illumination light beams may be emitted from the light sources, through the port, and into an optical fiber 106 partially housed inside probe 108.

System 100 delivers the laser and/or illumination light beams from the port to probe 108 via optical fiber 106. As shown, probe 108 includes a hand-piece, or probe body, 110. Probe 108 also includes a probe tip 140 coupled to a distal end of hand-piece 110. Note that, herein, a distal end of a component refers to the end that is closer to a patient's body, or where the laser and/or illumination light is emitted out of the probe. On the other hand, the proximal end of the component refers to the end that is facing away from the patient's body or in proximity to, for example, the light source. Probe tip 140 includes a tube 112 extending an entire length of probe tip 140. In certain embodiments, tube 112 is a cylindrical hollow tube. A distal end and a proximal end of probe tip 140 and thus, of tube 112, are depicted in FIG. 1. Although not shown herein, optical fiber 106 extends an entire length of tube 112 to transmit laser and/or illumination light to the distal end of tube 112.

In operation, a surgeon uses hand-piece 110 to guide tube 112 into a patient's eye 120. Tube 112 is only partly inserted into eye 120 such that the proximal end of tube 112 is disposed outside eye 120. A laser and/or illumination light source of surgical system 102 generates a light beam 150, which is directed by tube 112 to a desired location/surface of eye 120, such as retinal surface 122. In certain embodiments, probe 108 is a multi-spot laser probe and concurrently provides multiple laser light beams 150 resulting in multiple laser spots. Each laser spot's power may be within a range of about 150 milliwatts (mW) to about 500 mW such that by providing multiple laser spots, the minimum power passing through tube 112 is about 1 W (Watt). In certain embodiments, a lens is positioned in front of the one or more optical fibers in tube 112 for projecting the laser and/or illumination light beams onto the desired location of eye 120. Thus, as described above, system 100 is capable of projecting a laser and/or illumination light beam onto a desired location of the eye during a surgical procedure, e.g., retinal laser treatment.

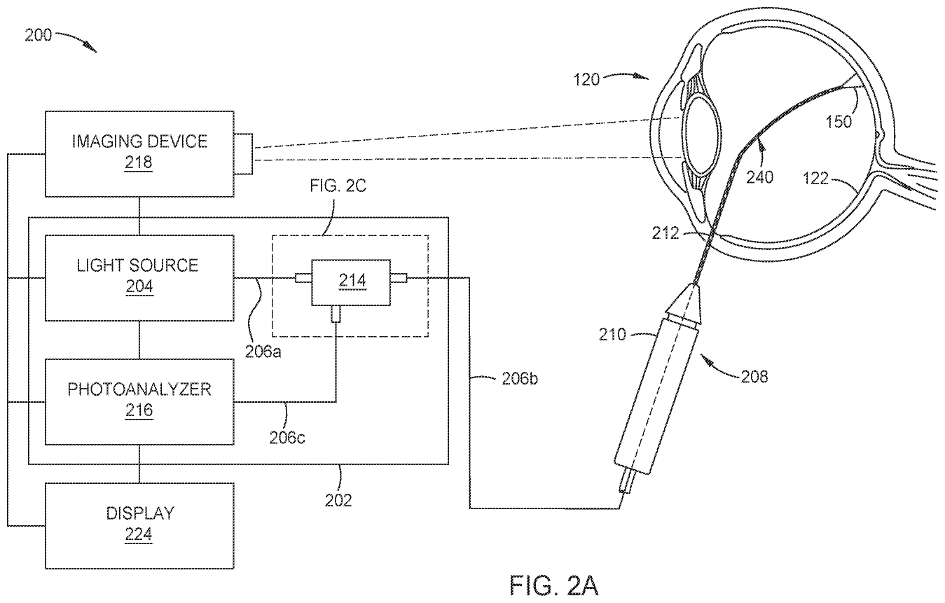

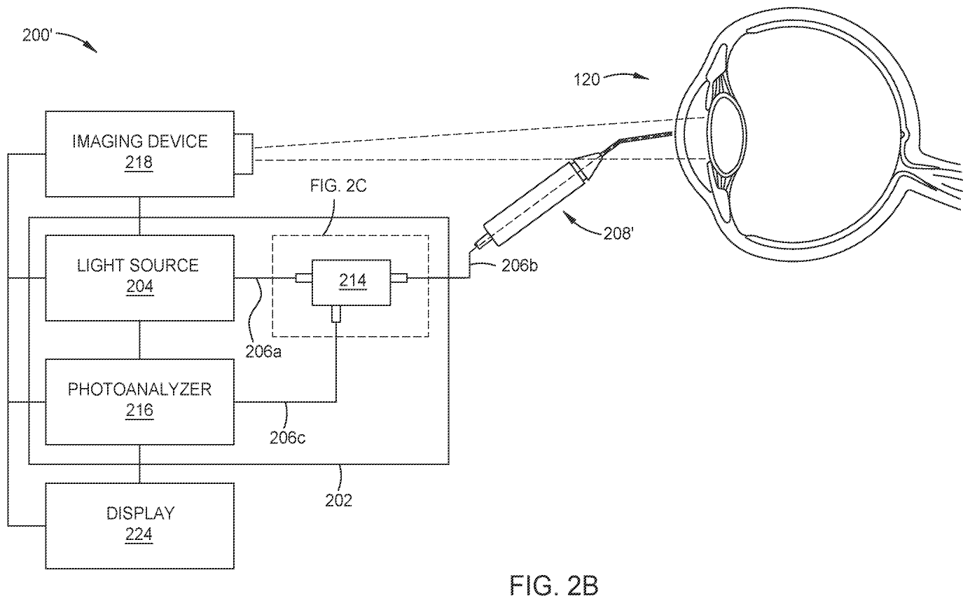

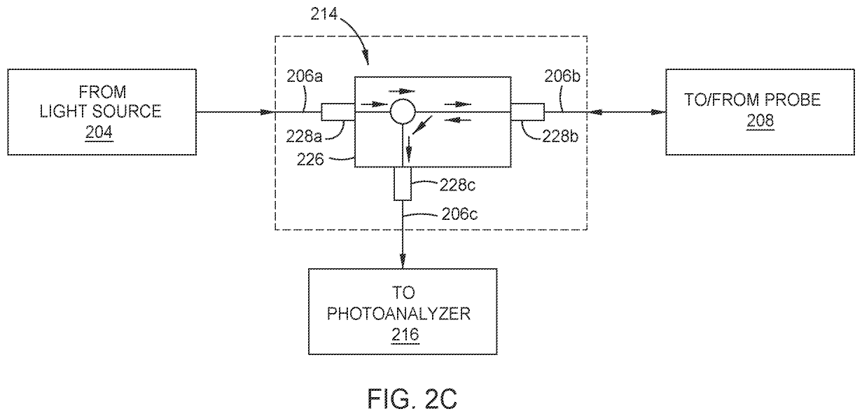

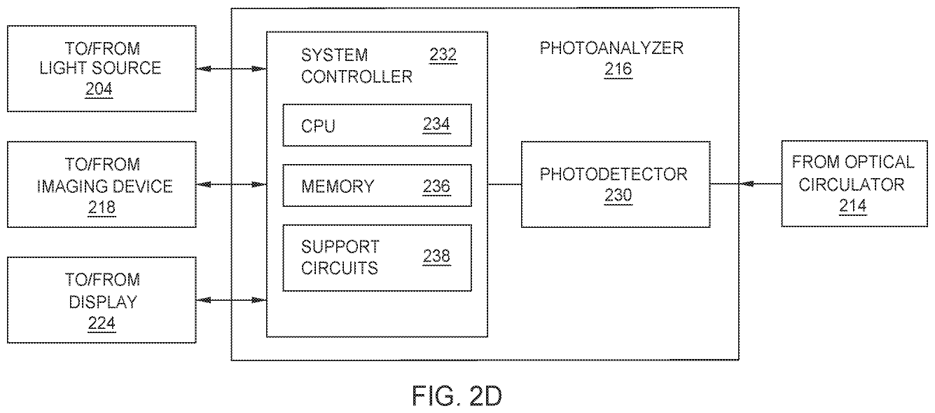

FIG. 2A illustrates an example optical system 200 for obtaining ophthalmic information, according to certain embodiments. Optical system 200 generally includes a surgical system 202 and a probe 208 coupled to surgical system 202. Surgical system 202 generally includes a light source 204, a photoanalyzer 216, and an optical circulator 214. As shown in FIG. 2A, optical circulator 214 is optically coupled to each of light source 204, photoanalyzer 216, and probe 208 through multiple optical fibers 206 (206a-c). In the illustrated embodiments, optical fiber 206a is coupled between light source 204 and optical circulator 214, optical fiber 206b is coupled between optical circulator 214 and probe 208, and optical fiber 206c is coupled between photoanalyzer 216 and optical circulator 214. Together, optical fibers 206a-c enable transmission of laser and/or illumination light from light source 204 to probe 208 and from probe 208 to photoanalyzer 216 as described in more detail below. Note that certain portions of optical fibers 206 may be disposed inside a cable. For example, a portion of optical fiber 206b located outside probe 208 may be disposed within an outer sleeve, whereas only the fiber without the outer sleeve is disposed inside probe 208. In certain embodiments, optical circulator 214 is directly coupled to one or both of light source 204 or photoanalyzer 216 such that optical system 200 may operate without one or both of optical fibers 206a or 206c. Directly coupling photoanalyzer 216 to optical circulator 214 may improve photodetection by reducing overall loss of the return light.

Light source 204 may be a laser source (coherent light source) and/or illumination source (incoherent light source). In certain embodiments, light source 204 is a xenon-based or LED-based illuminator. In certain embodiments, light source 204 is a broadband light source or hyperspectral light source. Hyperspectral light may include light beyond the visible spectrum including, e.g., infrared and ultraviolet light. Other light sources are also contemplated. For example, instead of a broadband light source, narrowband/discrete light source(s) (such as blue, green, red, etc. light) may be used for multispectral imaging. In certain embodiments, light source 204 is integrated with a console. In some other embodiments, light source 204 is a stand-alone light source. Optical circulator 214 is described in more detail below with respect to FIG. 2C. Photoanalyzer 216 includes a photodetector and a controller. Photoanalyzer 216 is described in more detail below with respect to FIG. 2D.

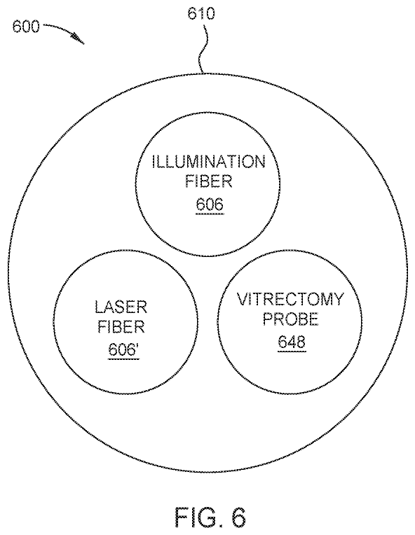

Probe 208 may be the same as or similar to probe 108 shown in FIG. 1. For example, probe 208 may generally include a hand-piece 210 and a probe tip 240 coupled to hand-piece 210. In certain embodiments, probe tip 240 is or includes a tube 212 for housing an optical fiber as described above. In certain embodiments, tube 212 is a cylindrical hollow tube. The gauge size and length of tube 212 may vary depending on the application. In certain embodiments, the gauge size ranges from 23 gauge to 27 gauge. Alternatively, probe 208 may have a different construction and/or operation compared to FIG. 1. For example, probe 208 is not limited to a light conduit probe. In certain embodiments, probe 208 is an image-preserving probe including, e.g., a gradient index fiber capable of relaying images of retinal surface 122 to optical circulator 214. In certain embodiments, probe 208 is an endoscopic probe with a camera at the distal end of probe tip 240. In some other examples, probe 208 is a light-emitting vitreous cutter (shown in FIG. 6) or another type of light-emitting surgical device. In certain embodiments, optical system 200 utilizes free space optics to transmit source light and return light. In such embodiments, source light and return light are transmitted without the use of optical fibers 206. In certain embodiments, probe 208 includes a return light detector that is separate from a source light output of probe 208. In certain embodiments, instead of a single probe 208, optical system 200 includes a first source light output probe and a second return light detector, which are independently insertable into eye 120.

In the illustrated embodiments, optical fiber 206b is disposed inside probe 208. In certain embodiments, optical fiber 206b may extend an entire length of probe 208 to transmit laser and/or illumination light therethrough. Optical system 200 is able to function as described herein when optical fiber 206b includes only one optical fiber. For example, a single optical fiber may transmit both laser light and illumination light. However, in some other embodiments, probe 208 includes two or more optical fibers to provide added functionality. In certain embodiments, a first optical fiber housed in probe 208 transmits an illumination light from a corresponding illumination source while a second optical fiber housed in probe 208 transmits a laser light from a corresponding laser source.

As shown in FIG. 2A, probe 208 is used to emit laser and/or illumination towards certain components inside eye 120. For example, probe 208 is partly inserted into eye 120 in order to target the posterior chamber. In the example of FIG. 2A, the optical system 200 obtains ophthalmic information by transmitting laser and/or illumination source light through probe 208, emitting the source light from probe tip 240 onto tissues/structures inside the eye, such as a targeted portion of the retinal surface, and then collecting return light (e.g, a portion of light reflected from the targeted tissues/structures) inside the probe 208 for subsequent analysis.

FIG. 2B illustrates another example optical system 200′ for obtaining ophthalmic information, according to certain embodiments. As shown in FIG. 2B, probe 208′ is not inserted into eye 120. Instead of emitting laser and/or illumination light from inside eye 120, probe 208′ emits laser and/or illumination light from outside eye 120. Probe 208′ is used to target tissues/structures that are visible in the front of eye 120. In certain embodiments, probe 208′ is pointed at the cornea to investigate conditions affecting the lens (e.g., cataracts). In the example of cataracts, typical practice only provides qualitative information regarding disease progression. The ophthalmic information obtained by probe 208′ may be used to quantify cataract progression in a clinical setting by tracking opacity of the lens based on light spectra of the return light, source light absorption, and/or source light scattering as described in more detail below. In certain embodiments, an imaging device generates hyperspectral images over multiple wavelengths of light emitted from probe 208′ to detect changes in retinal tissue reflectance. In some embodiments, the imaging device generates multispectral images (e.g., using narrowband light sources) over several, discrete wavelengths of light emitted from probe 208′ to detect changes in retinal tissue reflectance. An increased risk of retinal pathology with advancing age is associated with cataract formation, which affects the measured light spectrum thereby enabling disease diagnosis and monitoring based on retinal tissue reflectance. In addition, optical system 200′ may use signature matching to enhance the assessment of disease progression.

As shown in FIG. 2A, optical system 200 includes an imaging device 218 (e.g., a surgical microscope) coupled to surgical system 202. In the illustrated embodiments, surgical system 202 is separate from imaging device 218. In some other embodiments, surgical system 202 is electrically and/or physically integrated with imaging device 218. In general, imaging device 218 provides the surgeon with a two-dimensional or three-dimensional view of eye 120 during a procedure. In certain embodiments, imaging device 218 includes a digital camera for capturing an image that is transmitted to a display 224 for viewing. During a procedure on retinal surface 122 using optical system 200, the surgeon is able to visualize the posterior chamber of eye 120 along with the location of probe tip 240 with respect to retinal surface 122 using imaging device 218 and display 224.

A valuable benefit of optical system 200 is the ability to combine a global view of the eye using imaging device 218 with a much more localized view, which is provided by light returning from the eye as a result of laser or illumination light contacting a targeted eye tissue/structure that is captured by probe 208 as described in detail below. In other words, imaging device 218 is able to provide high-level, and in many cases only qualitative information, about eye tissues/structures, whereas probe 208 is able to provide much higher resolution, quantitative information related to a specific target area based on interaction of laser and/or illumination light with the targeted tissues/structures as described in more detail below.

In certain embodiments, imaging device 218 includes a hyperspectral camera that is able to provide a two-dimensional map of retinal surface 122. In certain embodiments, the two-dimensional map indicates degree of oxygenation/de-oxygenation of the retinal tissue that may be used by the surgeon in determining tissue patency when a patient is suffering from retinal detachment. In certain embodiments, use of a hyperspectral camera enables the surgeon to see through a retinal hemorrhage based on the transmission spectra of blood, thus improving visualization of retinal structures. In certain embodiments, use of a broadband light source, such as a broadband white LED enables measuring oxygenation/de-oxygenation levels in the blood. In particular, by monitoring spectral characteristics of the return light at wavelengths within a range of about 530 nm (nanometers) and 600 nm, blood oxygen levels may be determined and correlated to disease progression, such as for early detection of diabetic retinopathy. In some embodiments, a multispectral camera (or other camera types) may be used to provide a map of the retinal surface 122.

As shown in FIG. 2A, display 224 is coupled to photoanalyzer 216 and imaging device 218. Display 224 is capable of displaying to the surgeon information associated with photoanalyzer 216 and/or imaging device 218 including ophthalmic information obtained using probe 208. In the illustrated embodiments, display 224 is separate from imaging device 218. In some other embodiments, display 224 is integral with imaging device 218. In certain embodiments, display 224 includes an augmented reality display. In certain embodiments, display 224 includes a virtual reality display. In certain embodiments, display 224 includes a three-dimensional display to provide depth information to the surgeon.

FIG. 2C illustrates an example optical circulator 214 of optical system 200, according to certain embodiments. In general, optical circulators are capable of separating optical signals that travel in opposite directions in an optical fiber, thereby providing bi-directional transmission over a single optical fiber. As shown, optical circulator 214 generally includes a housing 226 and three ports 228 (228a-c). In some other embodiments, optical circulator 214 includes more than 3 ports. In certain embodiments, optical circulator 214 includes multiple glass tubes and polarization elements enclosed in housing 226 for controlling optical transmission therein. Optical circulator 214 enables transmission or coupling of both coherent light sources (e.g., lasers) and incoherent light sources (e.g., xenon-based and LED-based illuminators).

In certain embodiments, optical circulator 214 is a birefringent crystal based circulator in which circulation is realized through an arrangement of birefringent crystals, Faraday rotators, and beam displacers. In certain embodiments, light that enters and exits a birefringent crystal based circulator is collimated. In some other embodiments, optical circulator 214 is an optical fiber based circulator having a core and an inner cladding. Light that passes through an optical fiber based circulator in a first direction is transmitted through the core, whereas light that passes in the opposite direction is transmitted through the inner cladding. Other types of optical circulators may also be implemented in the optical systems disclosed herein.

As shown in FIG. 2C, laser and/or illumination light traveling in optical fiber 206a from light source 204 (referred to as “source light” or “forward light”) enters optical circulator 214 through port 228a. The source light entering port 228a exits optical circulator 214 through port 228b and is then transmitted to probe 208 through optical fiber 206b. The source light is then output from probe 208 in the form of light beam 150 (shown in FIG. 2A). Light beam 150 is projected onto retinal surface 122 of eye 120 to provide laser treatment and/or illumination to a target area. Upon contact with the targeted eye tissues/structures, light beam 150 is converted to or results in the formation of a return light that may include one or a combination of reflection, scattering, fluorescence, auto fluorescence, or Raman spectra components. At least a portion of the light returning from the eye as a result of light beam 150 contacting the targeted eye tissues/structures, is collected in optical fiber 206b. In certain embodiments, the return light includes at least a portion of light beam 150 that is reflected off retinal surface 122 (referred to as “reflected light”). The return light travels in optical fiber 206b in the opposite direction as the source light. The return light traveling in optical fiber 206b from probe 208 enters optical circulator 214 through port 228b. The return light entering port 228b exits optical circulator 214 through port 228c and enters optical fiber 206c. The return light is then output to photoanalyzer 216. Based on the return light, photoanalyzer 216 is able to determine one or more spectral parameters of the targeted eye tissues/structures, as well as other information, as described in detail below. In certain embodiments, the one or more spectral parameters include wavelength, frequency, wavenumber, and photon energy.

In certain embodiments, optical circulator 214 operates in near-visible wavelength range (e.g., 0.98 μm (micrometers), 1.3 μm, or 1.55 μm wavelength), visible wavelength range (e.g., 473 nm (blue laser), 532 nm (green laser), or 650 nm (red laser)), or broadband wavelength range (e.g., 300 nm to 700 nm). Optical circulator 214 may interface with single-mode or multi-mode optical fibers. Conventional optical circulators are built to couple to standard multi-mode optical fibers which may have a 50 μm core and 125 μm cladding, or alternatively, a 62.5 μm core and 125 μm cladding. In certain embodiments, optical fibers 206a-c have a 75 μm core and 90 μm cladding. Design of optical circulator 214 may be dependent upon the dimensions of optical fibers 206a-c. For example, dimensions of the glass tubes and polarization elements within optical circulator 214 may be customized to match the core and cladding dimensions of optical fibers 206a-c to enable optical coupling between optical fibers 206a-c and optical circulator 214.

FIG. 2D illustrates an example photoanalyzer 216 of optical system 200, according to certain embodiments. Photoanalyzer 216 generally includes a photodetector 230 for receiving the return light signals and a system controller 232 coupled to photodetector 230 for analyzing the return light signals in order to determine one or more spectral parameters of the targeted eye tissues/structures and other information. System controller 232 is shown as a part of photoanalyzer 216 in the illustrated embodiments. However, in some other embodiments, system controller 232 and photoanalyzer 216 are separate components.

Photodetector 230 may be operable to sense/detect various aspects of the return light signals including intensity and spectral information including, e.g., wavelength, polarization, and phase of the return light. In certain embodiments, photodetector 230 is a photodiode, avalanche photodiode, photomultiplier tube (PMT), or spectrometer.

System controller 232, such as a programmable computer, is coupled to photodetector 230. Based on the return signals received in photodetector 230, system controller 232 may be operable to characterize the return light spectra in terms of intensity, wavelength, polarization, phase, and spectral signatures. In certain embodiments, system controller 232 is able to determine optical distances based on time of flight data received in photodetector 230. In addition, system controller 232 may be operable to obtain data related to the interaction of the source light with the targeted eye tissues/structures, such as absorption and scattering of the source light within the eye.

In certain embodiments, system controller 232 is coupled to one or more of light source 204, imaging device 218, or display 224 for controlling optical system 200 or components thereof. For example, system controller 232 may control the operation of optical system 200 using a direct control of light source 204, imaging device 218, and/or display 224 or using indirect control of other controllers associated therewith. In operation, system controller 232 may enable data acquisition and feedback from the respective components to coordinate operation of optical system 200.

System controller 232 includes a programmable central processing unit (CPU) 234, which is operable with a memory 236 (e.g., non-volatile memory) and support circuits 238. Support circuits 238 are conventionally coupled to CPU 234 and comprise cache, clock circuits, input/output subsystems, power supplies, and the like, and combinations thereof coupled to the various components of optical system 200.

In some embodiments, CPU 234 is one of any form of general purpose computer processor used in an industrial setting, such as a programmable logic controller (PLC), for controlling various monitoring system component and sub-processors. Memory 236, coupled to CPU 234, is typically one or more of readily available memory, including volatile or non-volatile memory. For example, memory 236 may be a random access memory (RAM), read only memory (ROM), floppy disk drive, hard disk, or any other form of digital storage, local or remote.

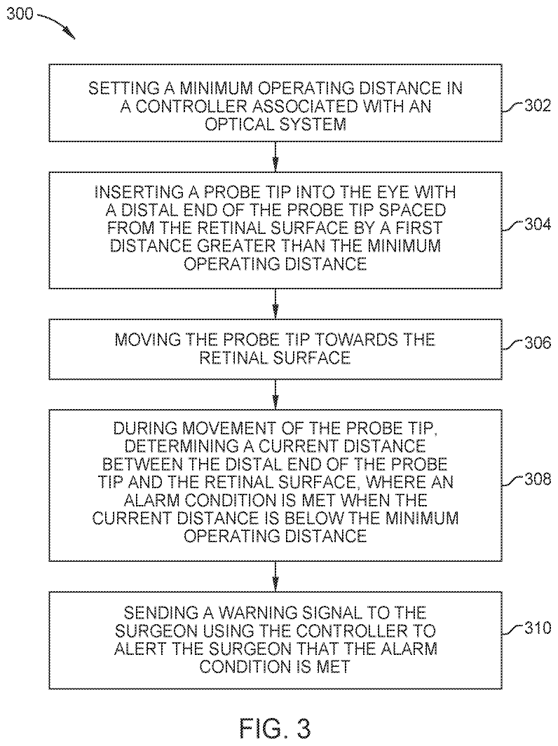

Herein, memory 236 stores instructions, that when executed by CPU 234, facilitates the operation of optical system 200 and/or photoanalyzer 216. The instructions in memory 236 are in the form of a program product such as a program that implements the methods of the present disclosure (e.g., middleware application, equipment software application, etc.). In certain embodiments, optical systems disclosed herein are able to determine a distance between a distal end of probe tip 240 and retinal surface 122 (which may be referred to as “ranging”). In certain embodiments, ranging is performed using pulses of laser and/or illumination light from light source 204. In certain embodiments, ranging measurements are based on time-of-flight (ToF) or the Doppler effect. FIG. 3 illustrates an example method 300 for using ranging data obtained by optical system 200 to improve safety during vitrectomy procedures (e.g., core vitrectomy or vitreous shaving), according to certain embodiments. Note that methods disclosed herein may be carried out using one or more of the optical system embodiments provided, and thus, optical system 200 is described in the following examples for illustrative purposes only.

Vitrectomy procedures are known to sometimes result in damage to the retinal surface due to inadvertent physical contact between the vitrectomy probe and the retina. Undesirable physical contact is especially prone to occur during vitreous shaving, which is performed in very close proximity to the retinal surface. Optical systems and/or methods disclosed herein are configured to reduce the occurrence of physical contact and associated retinal damage by alerting the surgeon when probe tip 240 is too close to retinal surface 122 as described in detail below. In vitrectomy applications, probe 208 includes a vitrectomy probe with an integrated optical fiber (shown in FIG. 6).

At operation 302, a minimum operating distance is set in a controller associated with optical system 200 (e.g., system controller 232). The minimum operating distance may correspond to a minimum working distance, or lower threshold, between the distal end of probe tip 240 and retinal surface 122 that the surgeon considers safe to continue the vitrectomy procedure. In certain embodiments, the minimum operating distance is set to about 2 mm (millimeters) or less, such as about 2 mm, or about 1 mm or less, such as about 1 mm. In certain embodiments, for laser probes and illuminated laser probes the minimum operating distance is about 2 mm. In certain embodiments, for an illuminated membrane pic or forcep, the minimum operating distance between a distal end of the pic or forcep and the retinal surface is about 1 mm. In some other embodiments, such as for an endoilluminator, the minimum operating distance is about 15 mm. In some other embodiments, such as for a wide angle, diffusing, or chandelier-type illuminator, the minimum operating distance is about 18 mm.

At operation 304, probe tip 240 is inserted into eye 120 as shown in FIG. 2A. In this position, the distal end of probe tip 240 is spaced from retinal surface 122 by a first distance greater than the minimum operating distance.

At operation 306, probe tip 240 is moved towards retinal surface 122. At operation 308, during movement of probe tip 240, photoanalyzer 216 determines a current distance between the distal end of probe tip 240 and retinal surface 122 based on the return light signals as described above with respect to FIGS. 2A-2C. Photoanalyzer 216 operates continuously to update the current distance in real-time. Using the controller, each current distance is compared to the minimum operating distance. An alarm condition is met when the current distance between the distal end of probe tip 240 and retinal surface 122 is below the minimum operating distance.

At operation 310, a warning signal or other indication is sent to the surgeon by a controller associated with optical system 200 (e.g., system controller 232) to alert the surgeon that the alarm condition is met. Based on the warning signal, the surgeon is able to make an informed decision either to continue the vitrectomy procedure below the minimum operating distance or to move probe tip 240 a greater distance away from retinal surface 122. Thus, as a result of the warning signal, safety of the vitrectomy procedure is improved.

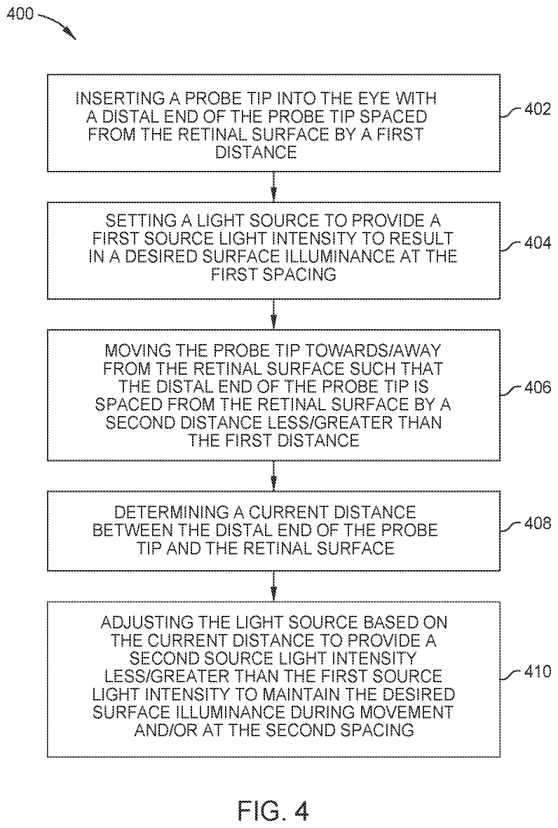

FIG. 4 illustrates another example method 400 for using ranging data obtained by optical system 200 to provide constant surface illuminance during a surgical procedure, according to certain embodiments. As used herein, the term “illuminance” refers to luminous flux per unit area.

At 402, probe tip 240 is inserted into eye 120 as shown in FIG. 2A. In this position, the distal end of probe tip 240 is spaced from retinal surface 122 by a first distance.

At 404, light source 204 is set to provide a first source light intensity to result in a desired surface illuminance at the first spacing.

At 406, probe tip 240 is moved towards/away from retinal surface 122. After probe tip 240 is moved, the distal end of probe tip 240 is spaced from retinal surface 122 by a second distance less/greater than the first distance. In this position, without adjusting light source 204, the surface illuminance increases above/decreases below the desired level which can reduce the surgeon's visibility of retinal surface 122 due to over/under exposure. Certain embodiments disclosed herein address the issue of reduced visibility by maintaining the surface illuminance at a constant level as described below.

At 408, photoanalyzer 216 determines a current distance between the distal end of probe tip 240 and retinal surface 122 based on the return light signals as described above with respect to FIGS. 2A-2C. Photoanalyzer 216 operates continuously to update the current distance in real-time.

At 410, light source 204 is adjusted based on the current distance to provide a second source light intensity less/greater than the first source light intensity to maintain the desired surface illuminance on retinal surface 122 when probe tip 240 is spaced from retinal surface 122 by the second distance. Operation 410 may occur simultaneously with movement of probe tip 240 at operation 406 such that adjustment of source light intensity occurs in real-time. In such embodiments, automated control of light source 204 using a controller associated with light source 204 and photoanalyzer 216 of optical system 200 (e.g., system controller 232) is capable of maintaining surface illuminance within a range where lighting changes are not noticeable to the surgeon. Alternatively, operation 410 may occur following movement of probe tip 240 at operation 406, such that light source 204 may simply be adjusted after probe tip 240 reaches the second spacing.

In some examples, the ranging data obtained by optical system 200 may be used in robotic applications. For example, the ranging data may be used in conjunction with end effector positioning data to improve control of the robotic system.

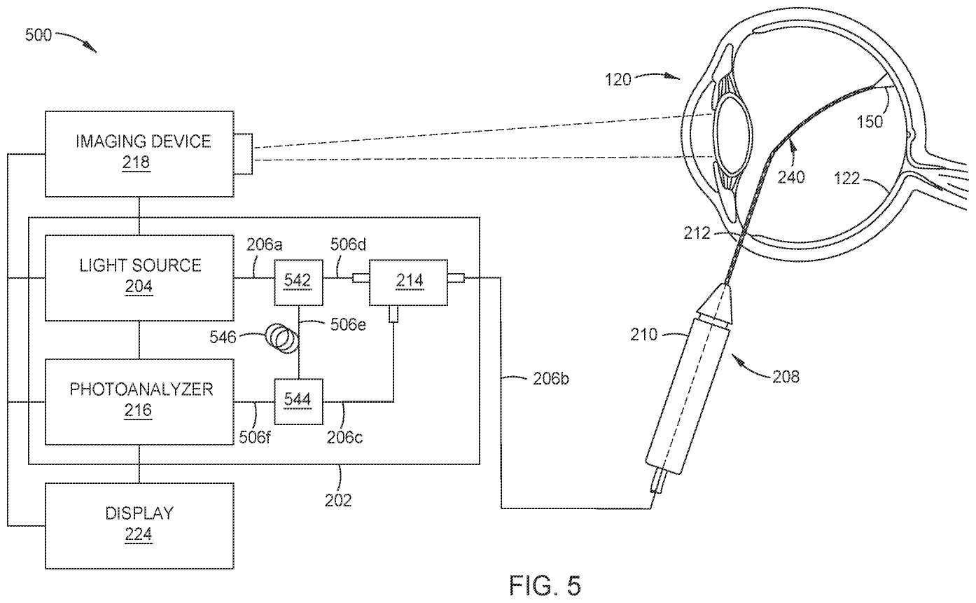

FIG. 5 illustrates yet another example optical system 500 for obtaining ophthalmic information, according to certain embodiments. Optical system 500 is the same as optical system 200 shown in FIG. 2A, except for the addition of a Michelson interferometer. In general, a Michelson interferometer enables the detection of an interference pattern between a reference beam and a measurement beam, which can provide additional information related to the object being measured, such as measurement of very small changes in distance and/or displacement. In FIG. 5, optical system 500 generally includes a fiber splitter 542 and a fiber coupler 544. Fiber splitter 542 is optically coupled between light source 204 and optical circulator 214. Fiber coupler 544 is optically coupled between optical circulator 214 and photoanalyzer 216.

Fiber splitter 542 has a single input and two outputs. The input of fiber splitter 542 is coupled to optical fiber 206a. A first output of fiber splitter 542 is coupled to optical fiber 506d. A second output of fiber splitter 542 is coupled to optical fiber 506e. Source light traveling in optical fiber 206a from light source 204 enters fiber splitter 542 through the input. Part of the source light entering the input exits fiber splitter 542 through each output. A first portion of source light passing from the input to the first output of fiber splitter 542 is transmitted to port 228a of optical circulator 214 through optical fiber 506d and is then transmitted to probe 208 as described above. The first portion of source light may be referred to as a “measurement beam.”

A second portion of source light passing from the input to the second output of fiber splitter 542 enters optical fiber 506e. The second portion of source light bypasses optical circulator 214. The second portion of source light may be referred to as a “reference beam.” The reference beam is subsequently combined with the return light portion of the measurement beam for coupling to photoanalyzer 216 as described in more detail below. In practice, optical fiber 506e includes a coil 546 for wrapping a long length of fiber, which matches a nominal fiber path length of the measurement beam to provide maximum interferometric coherence between the two combined beams.

In certain embodiments, fiber splitter 542 is a 50/50 splitter, which means that half of the source light enters optical fiber 506d through the first output and the other half of the source light enters optical fiber 506e through the second output.

Fiber coupler 544 has a two inputs and a single output. A first input of fiber coupler 544 is coupled to optical fiber 206c. A second input of fiber coupler 544 is coupled to optical fiber 506e. The output of fiber coupler 544 is coupled to optical fiber 506f. The return light collected in optical fiber 206b enters optical circulator 214 through port 228b and exits optical circulator 214 through port 228c. The return light is then transmitted to the first input of fiber coupler 544 through optical fiber 206c. The reference beam traveling in optical fiber 506e enters fiber coupler 544 through the second input. The return light and the reference beam are combined in fiber coupler 544 and the combined beam is transmitted to photoanalyzer 216 through optical fiber 506f. By sensing and analyzing an interference pattern between the reference beam and the return portion of the measurement beam, photoanalyzer 216 provides additional information related to the targeted eye tissues/structures. For example, photoanalyzer 216 may determine a distance between the distal end of probe tip 140 and retinal surface 122.

FIG. 6 illustrates an example illuminated vitrectomy cutter 600 that may be used herein, according to certain embodiments. Vitrectomy cutter 600 may be used in place of probe 208 or probe 208′ described above. Vitrectomy cutter 600 is shown in cross-section to schematically illustrate components thereof. Vitrectomy cutter 600 generally includes a housing 610 (e.g., hand-piece or probe tip), a vitrectomy probe 648 having a cutting head at a distal end thereof, a first optical fiber 606 (which may be referred to as “illumination fiber”) for illuminating locally near the cutting head, and an optional second optical fiber 606′ (which may be referred to as “laser fiber”) for transmitting laser light locally near the cutting head. Vitrectomy cutter 600 may be used to monitor and optimize the cutting process by analyzing return light collected in first optical fiber 606.

In summary, embodiments of the present disclosure enable the acquisition of medical information during surgical procedures using an optical system including a laser and/or illumination probe. Certain embodiments provide valuable information in multiple types of procedures such as retinal surgery, cataract surgery, diagnostic procedures (e.g., diagnosis of dry eye and glaucoma), and other surgical procedures as well as in the detection of disease conditions (e.g., retinal blastoma). Optical systems and/or methods described herein are particularly advantageous for personalizing laser treatments and/or improving treatment results, for improving the safety and/or effectiveness of surgical procedures, for providing additional data points to improve diagnostic accuracy, and for detecting key spectral signatures associated with certain disease conditions, among many other benefits described above.

While the foregoing is directed to embodiments of the present disclosure, other and further embodiments of the disclosure may be devised without departing from the basic scope thereof, and the scope thereof is determined by the claims that follow.

What is claimed is:

1. An optical system, comprising:
a probe housing a first optical fiber;
a light source;
a photoanalyzer; and
an optical circulator optically coupled to each of the first optical fiber, the light source, and the photoanalyzer, the optical circulator comprising:
a first port configured to receive source light generated from the light source;
a second port configured to transmit the source light from the first port to the first optical fiber, wherein the first optical fiber is configured to:
emit at least a portion of the source light in the first optical fiber from the probe to contact a body structure, and
collect light returning from the body structure as a result of the portion of the source light contacting the body structure; and
a third port configured to transmit the return light in the first optical fiber from the second port to the photoanalyzer, the photoanalyzer configured to determine one or more spectral parameters of the body structure based on the return light.

2. The optical system of claim 1, wherein the light source comprises at least one of a laser source, a broadband illumination source, a hyperspectral illumination source, or a multispectral illumination source.

3. The optical system of claim 1, wherein the one or more spectral parameters comprise wavelength, frequency, wavenumber, or photon energy.

4. The optical system of claim 1, wherein the photoanalyzer comprises:
a photodetector for receiving the return light; and
a processor for determining the one or more spectral parameters based on the return light received in the photodetector.

5. The optical system of claim 1, wherein the return light comprises at least one of reflection, scattering, fluorescence, auto fluorescence, Raman spectra, or combinations thereof.

6. The optical system of claim 1, wherein the one or more spectral parameters are determined based on at least one of light spectra of the return light, source light absorption, or source light scattering.

7. The optical system of claim 6, wherein:
a light spectrum of the return light comprises at least one of intensity data, wavelength data, polarization data, phase data, or time of flight data, and
the one or more spectral parameters are determined based on the light spectrum of the return light.

8. The optical system of claim 1, further comprising:
a second optical fiber coupled between the light source and the first port of the optical circulator; and
a third optical fiber coupled between the third port of the optical circulator and the photoanalyzer.

9. The optical system of claim 1, wherein the probe comprises a vitreous cutter.

10. The optical system of claim 1, wherein the light source comprises an illumination source, the optical system further comprising:
a laser source; and
a second optical fiber housed in the probe, wherein laser light from the laser source is transmitted in the second optical fiber.

11. The optical system of claim 1, further comprising a memory storing executable instructions that cause the system to:
determine a distance between a distal end of the probe and the body structure based on the return light; and
generate an indication if the determined distance is below a threshold.

12. The optical system of claim 1, wherein the light source is a laser source, further comprising a memory storing executable instructions that cause the system to:
perform laser treatment on the body structure; and
adjust at least one of a power, pulse duration, pulse frequency, or treatment time of the laser treatment based on the one or more spectral parameters of the body structure.

13. The optical system of claim 1, further comprising a memory storing executable instructions that cause the system to:
determine a composition of fluid inside the body structure.

14. The optical system of claim 1, further comprising a Michelson interferometer.

15. The optical system of claim 1, further comprising:
a fiber splitter optically coupled between the light source and the optical circulator, the fiber splitter comprising:
an input configured to receive the source light generated from the light source;
a first output configured to transmit a first portion of the source light from the input to the first port of the optical circulator; and
a second output configured to transmit a second portion of the source light from the input bypassing the optical circulator, wherein:
the second portion of the source light corresponds to a reference signal, and
the second portion of the source light is combined with the return light for transmission to the photoanalyzer.
